(19) United States
(12) Patent Application Publication
(10) Pub. No.: US 2011/0189698 A1
(43) Pub. Date: Aug. 4, 2011

(54) Protein Biomarkers and Methods for Diagnosing Kawasaki Disease

(75) Inventor: Jane C. Burns, La Jolla, CA (US)

(73) Assignee: THE REGENTS OF THE UNIVERSITY OF CALIFORNIA, Oakland, CA (US)

(21) Appl. No.: 13/036,751

(22) Filed: Feb. 28, 2011

Related U.S. Application Data
(63) Continuation of application No. PCT/US2009/055410, filed on Aug. 28, 2009.
(60) Provisional application No. 61/092,451, filed on Aug. 28, 2008.

(51) Int. Cl. G01N 33/68; G01N 33/74; G01N 33/573; G01N 30/00

(52) U.S. Cl. 435/7.4; 436/501; 435/7.1; 422/69

(57) ABSTRACT

A method, kit and device for diagnosing Kawasaki Disease are provided. The invention provides detecting an expression level of at least two Kawasaki Disease diagnostic biomarkers in a biological sample from a patient with a capture agent and diagnosing the patient as having Kawasaki Disease when the expression levels of the at least two diagnostic biomarkers in the patient biological sample are higher than the normal expression levels of the same biomarkers in a biological sample from a control subject. The first Kawasaki Disease diagnostic biomarker disclosed in the present invention is a cardiomyocyte biomarker, and the second Kawasaki Disease diagnostic biomarker is an inflammatory biomarker. The invention further provides detecting an expression level of a third biomarker, interferon type-I biomarker, in the patient biological sample with a capture agent and diagnosing the patient as having Kawasaki Disease when the expression level of interferon type-I biomarker is lower than the expression level in a control subject.

### CROSS-REFERENCE TO RELATED APPLICATIONS

This patent application is a continuation of PCT Application No. PCT/US09/055,140, filed Aug. 28, 2009, which claims priority benefit of U.S. Provisional Application No. 61/092,451 filed Aug. 28, 2008, each of which is incorporated herein by reference in their entireties.

### STATEMENT OF GOVERNMENT INTEREST

This invention was made with government support under NHLBI R01-HL69413 awarded by National Institutes of Health. The government has certain rights in the invention.

### FIELD OF THE INVENTION

The invention relates generally to the field of diagnosis of Kawasaki Disease (KD).

### BACKGROUND OF THE INVENTION

Various publications, including patents, published applications, technical articles and scholarly articles are cited throughout the specification. Each of these cited publications is incorporated by reference herein, in its entirety.

Kawasaki Disease (KD) is a self-limited acute vasculitis, the cause of which remains unknown. KD primarily affects infants and young children under five years old, although cases have been reported in older children and adolescents (Rosenfeld et al. (1995) J. Pediatr. 126:524-9; and, Stockheim et al. (2000) J. Pediatr. 137:250). It is the leading cause of acquired heart disease in children. Depending on the region, the average annual incidence varies from 9 cases per 100,000 population in the US to 200 cases per 100,000 children in Japan. It is believed, however, that the incidence of KD is considerably higher, the relatively low apparent incidence attributable primarily to misdiagnosis, as diseases such as adenovirus and streptococcal infections can mimic the symptoms of KD (Barone et al. (2000) Arch. Pediatr. Adolesc. Med. 154:453-6; and, Burns et al. (1991) J. Pediatr. 118:680-6), and to the lack of an acceptable diagnostic test. A recurrence rate of 3-5% has been reported in Japan.

KD patients present with prolonged fever, rash, swelling and redness of the hands and feet, conjunctival injection, redness of the mucous membranes of the mouth, lips, and throat, and lymphadenopathy (Burns et al. (2004) Lancet 364:533-44). It causes inflammation of the child's blood vessels. Symptoms generally abate on their own after about two to three weeks, and the disease is effectively treated with intravenous immunoglobulin (IVIG) (Newburger et al. (1986) N. Engl. J. Med. 324:1633-9). Efficacy of treatment, however, requires prompt diagnosis and prompt administration of IVIG. If not detected and treated immediately, it can result in heart damage and death.

Although KD is self-limiting, failure to administer timely treatment with IVIG results in a significant risk that the KD patient will develop coronary artery disease. In fact, coronary artery aneurysms are observed in approximately 25% of untreated KD patients (Burns et al. (2004) Lancet 364:533-44). Moreover, there is an increased risk of coronary aneurysm for patients who have had prolonged KD or are afflicted at less than one year of age. The development of such aneurysms is often clinically silent, and may go unrecognized until a sudden death or myocardial infarction later in adult life (Burns et al. (1996) J. Am. Coll. Cardiol. 28:253-7). KD is recognized as the leading cause of acquired heart disease in children in the United States (Newburer et al. (2004) Curr. Opin. Pediatr. 16:508-14). Early treatment is effective in preventing the acquired heart disease, but first KD must be recognized in a timely manner.

However, not only is the cause of KD not known, there is no diagnostic test available to this date. The current diagnosis is solely based on clinical signs. As the etiology of KD remains elusive, current diagnosis is primarily by way of guidelines set by the American Heart Association, including a prolonged fever lasting at least 5 days, and the overall clinical picture that occurs over the following stages: (1) acute febrile phase (days 1-11); (2) subacute phase (days 11-21); (3) convalescent phase (days 21-60); and, (4) chronic phase in those individuals that develop cardiac complications (Newburger et al. (2004) Circulation 110:2747-71).

The current diagnosis criteria for KD has severe limitations: a) not all clinical signs may be present at any point in time, b) patients may be seen by different doctors on different days, c) atypical KD patients exist who do not manifest all the clinical signs but who go on, nonetheless, to develop coronary artery aneurysms which are a potentially fatal complication of the vasculitis. Awaiting the observation of clinical signs can result in missing the critical window for IVIG treatment, thereby making the patient susceptible to coronary artery damage.

In addition, early diagnosis would help differentiate KD patients from children with other rash/fever illnesses, and permit treatment and monitoring of children in the early stages of KD. Thus, more rapid, accurate, and reliable means for the diagnosis of KD that are minimally invasive and can be readily administered to all patients suspected of having KD are needed.

Attempts to identify a single biomarker for KD have not been successful, as the markers identified lacked sensitivity and/or lacked specificity for KD. For example, elevated levels of matrix metalloproteinase 9 (MMP-9) and tissue inhibitor of metalloproteinase 1 (TIMP-1) (Chua P K et al. (2003) Clin. Diagn. Lab. Immunol. 10:308-14; Gavin et al. (2003) Arterioscler. Thromb. Vasc. Biol. 23:576-81; and, Takeshita et al. (2001) Clin. Exp. Immunol. 125:340-4), members of the 5100 family of calcium-binding proteins (Ebihara et al. (2005) Eur. J. Pediatr. 164:427-31; Ye et al. (2004) Am. J. Cardiol. 94:840-4; and, Foell et al. (2003) Lancet 361:1270-2), serum brain natriuretic peptide (BNP), and D-dimer concentrations were observed in KD patients (Imamura et al. (2005) Eur J. Pediatr. 164:526-7: Kurotobi et al. (2005) Pediatr. Cardiol. 26:425-30; and, Kawamura et al. (2000) Pediatr. Int. 42:241-8). As these and other markers studied for their association with KD lack specificity, the need exists to identify a specific test to diagnose KD.

Genome-wide analyses of gene expression patterns offer the possibility of discovering disease-specific pathogenic processes in a relatively comprehensive and un-biased manner. This approach has been used extensively to study cancer, and more recently in the field of infectious disease (Liu et al. (2006) Curr Opin Microbiol. 9:312-9). Whole-blood RNA was analyzed to characterize the response to hemorrhagic viruses in nonhuman primates, malaria in Kenyan children, and identify patterns of gene expression associated with the development of shock in patients with dengue virus infection. Whole blood transcript patterns in KD patients, and identified a highly dynamic pattern of transcription associated with the acute phase of the disease, as well as specific transcript levels associated with the risk of subsequently failing to respond to IVIG therapy were also studied (Jeffrey et al. (2006) Nat. Immunol. 7:274-83).

### SUMMARY OF THE INVENTION

The invention features a method, kit, and device for diagnosing Kawasaki Disease, and providing a diagnostic biomarker panel for differentiating acute Kawasaki Disease from a non-specified viral infection. The diagnostic biomarker panel disclosed in the present invention includes at least two biomarkers associated with Kawasaki Disease. In certain embodiments, the methods disclosed in the present invention comprise the steps of a) detecting levels of at least two Kawasaki Disease diagnostic biomarkers in a biological sample from the patient, wherein the first biomarker is a cardiomyocyte biomarker, and the second biomarker is an inflammatory biomarker, and b) diagnosing the patient as having Kawasaki Disease when the levels of the at least two diagnostic biomarkers in the patient sample are higher than normal levels of the same biomarkers derived from a biological sample from a control subject without Kawasaki Disease.

In certain embodiments, the diagnostic biomarker panel provided by the present invention comprises a cardiomyocyte biomarker in combination with an inflammatory biomarker. Alternatively, the diagnostic biomarker panel provided by the present invention comprises at least two cardiomyocyte biomarkers; or at least two inflammatory biomarkers; or combinations thereof.

As used herein, the cardiomyocyte biomarker includes, but is not limited to, N-terminal pro-B-type natriuretic peptide (NT-proBNP), C-type natriuretic peptide (CNP), atrial natriuretic peptide (ANP), high-sensitivity Troponin, Interleukin-33 (IL-33), or ST2.

As used herein, the inflammatory biomarker includes, but is not limited to, a biomarker of endothelial cell, platelet, and leukocyte damage, activation and adhesion, a biomarker of macrophage monocytes, or a biomarker of acute phase reactants. As used herein, the biomarker of endothelial cell, platelet, and leukocyte damage, activation and adhesion includes, but is not limited to, vascular endothelial growth factor (VEGF), CD40, intercellular adhesion molecule-1 (ICAM-1), vascular cell adhesion molecule-1 (VCAM-1), carcinoembryonic antigen cell adhesion molecule-1 (CEACAM-1), Endogenous Secretory-Receptor for advanced glycation end products (EN-RAGE), pro-calcitonin, calcitonin, Interleukin-6 (IL-6), Interleukin-4 (IL-4), myeloperoxidase (MPO), osteoprotegrin (OPG), neutrophil elastase, matrix metalloproteinase-8 (MMP-8), Secreted Protein And Rich in Cysteine (SPARC), junctional adhesion molecule 3 (JAM3), spermine oxidase (SMOX), Cell-matrix adhesion intergrin molecule ITGB5 and ITGA2B, endothelin-1, signal peptide-CUB-EGF-like domain containing protein 1 (SCUBE1). As used herein, the biomarker of macrophage monocytes includes, but is not limited to, S100, S100A6, S100A8, S100A9, S100A11, S100A12, S100A13, S100P, S100Z, Macrophage inflammatory protein-1 alpha (MIP-1 alpha), Tissue inhibitor of matrix metalloproteinase-1 (TIMP-1). As used herein, the biomarker of acute phase reactants include, but is not limited to, alpha-1 antitrypsin, C-reactive protein, and fibrinogen.

The present invention also provides that the at least two biomarkers in the disclosed diagnostic biomarker panel can be any cardiomyocyte or inflammatory biomarkers now known or later determined to be associated with Kawasaki Disease. The present invention further provides that the expression levels of these Kawasaki Disease diagnostic biomarkers in a biological sample from a patient can be detected using a capture agent, and then compared to the reference values for the same biomarkers in healthy subjects. The reference values to which the detected values are compared can be those established for patients positive for Kawasaki Disease, for patients negative for Kawasaki Disease, or both. A change in the expression level of the at least two biomarkers in a biological sample from the patient relative to the reference values indicates whether the patient is or is not afflicted with Kawasaki Disease.

The present invention further provides that the capture reagent can be any organic or inorganic chemical, biomolecule, or any fragment, homolog, analog, conjugate, or derivative thereof, that specifically interacts with the Kawasaki Disease diagnostic biomarkers. In certain embodiments, the capture reagent is a protein or antibody that specifically detects the Kawasaki Disease diagnostic biomarkers in the diagnostic biomarker panel. In other embodiments, the capture agent is an oligonucleotide that binds to biomarker oligonucleotide RNA or DNA. In yet certain other embodiments, the capture reagent can be coupled to a solid support. In yet certain embodiments, the biological samples from the patient are biological fluid, including, but not limited to, whole blood, plasma, serum, tears, saliva, mucous, cerebrospinal fluid, or urine.

The method disclosed in the present invention further comprises a step of detecting an expression level of a third biomarker in a biological sample from the patient. As used herein, the third biomarker includes, but is not limited to, an interferon type-I biomarker. The present invention provides that the patient is diagnosed as having Kawasaki Disease when the expression level of the interferon type-I biomarker is lower in the patient sample than in a sample from a control subject without Kawasaki Disease. Detecting expression levels of additional interferon Kawasaki Disease diagnostic biomarkers, now known or later discovered, in the diagnostic panel, and evaluating changes in the detected expression levels of these biomarkers relative to the reference values for diagnosing a patient as having Kawasaki Disease are also within the scope of the present invention.

The present invention further provides a kit for diagnosing Kawasaki Disease comprising a capture reagent comprising a) one or more detectors specific for at least two Kawasaki Disease diagnostic biomarkers, wherein the first biomarker is a cardiomyocyte biomarker, and the second biomarker is an inflammatory biomarker, b) a detection reagent, and c) instructions for using the kit to diagnose a patient as having Kawasaki Disease when the expression levels of the at least two diagnostic biomarkers in a patient test sample are higher than the expression levels of the same biomarkers in a control subject without Kawasaki Disease. In certain embodiments, the cardiomyocyte biomarker includes, but is not limited to, N-terminal pro-B-type natriuretic peptide (NT-proBNP), C-type natriuretic peptide (CNP), atrial natriuretic peptide (ANP), high-sensitivity Troponin, IL-33, or ST2, and the inflammatory biomarker includes, but is not limited to, a biomarker of endothelial cell, platelet, and leukocyte damage, activation and adhesion, a biomarker of macrophage monocytes, and a biomarker of acute phase reactants, as discussed above.

The present invention further provides a kit that comprises a capture reagent comprising a detector that specifically detects an interferon type-I biomarker, and instructions for using the kit to diagnose a patient as having Kawasaki Disease when the expression level of the interferon type-I biomarker is lower in patient biological sample than in a control subject without Kawasaki Disease. A kit comprising other capture reagents that comprise detectors specifically detect other additional interferon Kawasaki Disease diagnostic biomarkers, now known or later discovered, and instructions to diagnose a patient as having Kawasaki Disease when the expression levels of these diagnostic biomarkers in patient are different from the expression levels of the same biomarkers in a control subject without Kawasaki Disease is also within the scope of the present invention.

The kits of the present invention can further comprise appropriate positive and negative controls against which a biological sample from a patient can be compared. The kits can further comprise ranges of reference values established for the expression of Kawasaki Disease patients positive for Kawasaki Disease, for patients negative for Kawasaki Disease, or both.

Also featured are devices to diagnose Kawasaki Disease. The Kawasaki Disease diagnostic device disclosed in the present invention comprises a) a capture reagent comprising one or more detectors specific for at least two Kawasaki Disease diagnostic biomarkers, wherein the first biomarker is a cardiomyocyte biomarker, and the second biomarker is an inflammatory biomarker, and b) detecting reagents for detecting an expression level of the at least two diagnostic biomarkers in a biological sample from the patient, wherein the patient is diagnosed as having Kawasaki Disease when the expression levels of the at least two diagnostic biomarkers in the patient biological sample are higher than the expression levels in a control subject without Kawasaki Disease. The capture reagent of the devices can be an organic or inorganic chemical, biomolecule, or any fragment, homolog, analog, conjugate, or derivative thereof that specifically interacts with the Kawasaki Disease diagnostic biomarkers. In certain embodiments, the capture reagent is a protein and/or an antibody, and may be immobilized on a solid support.

The device of the present invention also comprises a) a capture reagent comprising a detector specific for an interferon type-I biomarker, and b) detecting reagents for detecting an expression level of the interferon type-I biomarker, wherein the patient is diagnosed as having Kawasaki Disease when the expression level of the interferon type-I biomarker is lower in patient biological sample than in a control subject without Kawasaki Disease. The device of the present invention can also comprises a capture reagent comprising one or more detector for other Kawasaki Disease diagnostic biomarkers, now known or later discovered, and detecting reagents for detecting expression levels of these Kawasaki Disease diagnostic biomarkers in a biological sample from a patient, wherein the patient is diagnosed as having Kawasaki Disease when the expression levels of these diagnostic biomarkers in patient biological sample are different from the expression levels of the same biomarkers in a control subject without Kawasaki Disease.

The present invention further provides a diagnostic biomarker panel that differentiates between pediatric patients with fever due to non-specified viral infection versus acute Kawasaki disease, which requires immediate hospitalization and treatment to prevent permanent heart damage. The present invention provides the first time the diagnostic biomarker panel comprising Kawasaki Disease diagnostic biomarkers for differentiating these two types of patients, and/or identifying patients with a highest risk for complications of Kawasaki Disease, including coronary aneurysms. Identifying children at risk for coronary artery complications of Kawasaki Disease will lead to more specific, aggressive therapies for this subpopulation of Kawasaki Disease children.

In certain embodiments, the present invention provides a diagnostic method, kit, and device to determine which children with Kawasaki Disease are at greatest risk for coronary aneurysms or adverse outcomes from the disease. A patient's body fluid, including blood, saliva, urine, or tears, is subjected to an assay to detect at least 2, 3, 4, 5, 6, or more biomarkers associated with Kawasaki Disease. Each biomarker has a routinely determinable cut-off point that differentiates between Kawasaki Disease and febrile controls through manual or computer-assisted determination. In particular, the combination of biomarkers results greatly increases the accuracy of the diagnosis. In certain embodiments, the assays are separated by standard ELISAs. In alternative embodiments, a point-of-service assay utilizing technology with different monoclonal antibodies adhered to signaling systems is performed to capture biomarker proteins of interest.

The present invention provides a diagnostic method, kit, and device for diagnosing Kawasaki Disease that would be used widely in pediatric offices and emergency rooms to diagnose or rule out Kawasaki Disease in children with fever and rash. Thus, the universe of children who would be tested is much larger than the universe of children with Kawasaki Disease.

### BRIEF DESCRIPTION OF THE DRAWINGS

FIGS. 1(A) and 1(B) illustrate plasma concentrations of NT-proBNP and ST2 in KD and control subjects, respectively. Box plots show median, interquartile range, and 5th and 95th percentiles. FIG. 1(C) illustrates a scatterplot between NT-proBNP and ST2 in acute KD subjects with both results for both tests (n=57). ST2 and NTproBNP were modestly correlated. For panels A and B: p<0.00001 Kruskal-Wallis test for differences among groups; Pairwise comparison by Mann Whiney U test: p<0.05 for acute KD vs. FC for both NT-proBNP and ST2. Abbr: Cony KD=convalescent phase Kawasaki disease; FC=febrile control; HC=healthy control; KD=Kawasaki disease; NT-proBNP=N-terminal propeptide B-type natriuretic peptide.

FIG. 2 illustrates a Receiver-Operator Characteristic curve for NTproBNP in acute KD versus Febrile Control patients. As an example from this curve, with a cutpoint set at 1,809 pg/ml, test values above that level would have 71% sensitivity and 72% specificity in identifying patients with acute KD.

FIG. 3 illustrates a Receiver-Operator Characteristic curve for ST2 in acute KD versus Febrile Control patients. As an example from this curve, with a cutpoint set at 33.7 U/ml, test values above that level would have 55% sensitivity and 71% specificity in identifying patients with acute KD.

FIG. 4 illustrates interferon-responsive gene transcript levels. Transcript levels in whole blood were measured using TaqMan real-time reverse transcription polymerase chain reaction and normalized to levels of TAF1B transcript. * P<0.05

FIG. 5 illustrates an average expression of gene clusters associated with Kawasaki Disease (KD).

FIG. 6 illustrates a comparison of whole-blood transcription profiles from patients with Kawasaki disease (KD) and control groups. Open circle, data point outside the 90^{th} percentile. P values were calculated with the nonparametric Spearman rank sum test; NS, not significant, GAS, group A β-hemolytic Streptococcus infection; Rxn, reaction.

FIG. 7 illustrates serum concentration of biomarker alpha-I antitrypsin in acute KD and FC.

FIG. 8 illustrates serum concentration of biomarker calcitonin in acute KD and FC.

FIG. 9 illustrates serum concentration of biomarker CD40 in acute KD and FC.

FIG. 10 illustrates serum concentration of biomarker C Reactive Protein in acute KD and FC.

FIG. 11 illustrates serum concentration of biomarker EN-RAGE in acute KD and FC.

FIG. 12 illustrates serum concentration of biomarker Erythropoietin in acute KD and FC.

FIG. 13 illustrates serum concentration of biomarker Fibrinogen in acute KD and FC.

FIG. 14 illustrates serum concentration of biomarker ICAM-1 in acute KD and FC.

FIG. 15 illustrates serum concentration of biomarker IL-6 in acute KD and FC.

FIG. 16 illustrates serum concentration of biomarker MIP-1 alpha in acute KD and FC.

FIG. 17 illustrates serum concentration of biomarker Myeloperoxidase in acute KD and FC.

FIG. 18 illustrates serum concentration of biomarker TIMP-1 in acute KD and FC.

FIG. 19 illustrates serum concentration of biomarker VEGF in acute KD and FC.

### DETAILED DESCRIPTION OF THE INVENTION

In accordance with the present invention, it has been discovered that certain markers associated with Kawasaki Disease that are individually not sufficient for a diagnosis of Kawasaki Disease or otherwise lack sufficient sensitivity for reliable Kawasaki Disease detection can nevertheless be accurately indicative of Kawasaki Disease when analyzed in the aggregate or in a panel, and can thus form the basis for a rapid and reliable diagnostic test for the presence of Kawasaki Disease. Accordingly, the present invention features a method, kit and device to diagnose Kawasaki Disease in a patient, and to provide a diagnostic panel to differentiate acute Kawasaki Disease from non-specified viral infection and other disease conditions.

The inventive method disclosed in the present invention comprises the steps of: a) detecting expression levels of at least two Kawasaki Disease diagnostic biomarkers in a biological sample from the patient, wherein the first biomarker is a cardiomyocyte biomarker, and the second biomarker is an inflammatory biomarker, and b) diagnosing the patient as having Kawasaki Disease when the expression levels of the at least two diagnostic biomarkers in the patient sample are higher than normal levels of the same biomarkers derived from a biological sample from a control subject without Kawasaki Disease.

In certain embodiments, the present invention provides a diagnostic biomarker panel for diagnosing and distinguishing acute Kawasaki Disease from other non-specific viral infection comprising one or more cardiomyocyte biomarker in combination with one or more inflammatory biomarkers. Alternatively, the diagnostic biomarker panel provided by the present invention comprises at least two cardiomyocyte-biomarkers; or at least two inflammatory biomarkers; or combinations thereof.

As used herein, the cardiomyocyte biomarker includes, but is not limited to, N-terminal pro-B-type natriuretic peptide (NT-proBNP), C-type natriuretic peptide (CNP), atrial natriuretic peptide (ANP), high-sensitivity Troponin, IL-33, or ST2. In preferred embodiments, the cardiomyocyte biomarker is NT-proBNP. In yet other preferred embodiments, the cardiomyocyte biomarker is ST2.

As used herein, the inflammatory biomarker includes, but is not limited to, a biomarker of endothelial cell, platelet, and leukocyte damage, activation and adhesion, a biomarker of macrophage monocytes, and a biomarker of acute phase reactants. As used herein, the biomarker of endothelial cell, platelet, and leukocyte damage, activation and adhesion includes, but is not limited to, vascular endothelial growth factor (VEGF), CD40, intercellular adhesion molecule-1 (ICAM-1), vascular cell adhesion molecule-1 (VCAM-1), carcinoembryonic antigen cell adhesion molecule-1 (CEACAM-1), EN-RAGE, pro-calcitonin, calcitonin, IL-6, IL-4, myeloperoxidase (MPO), osteoprotegrin (OPG), neutrophil elastase, MMP-8, SPARC, JAM3, SMOX, ITGB5, ITGA2B, endothelin-1, SCUBE1. As used herein, the biomarker of macrophage monocytes includes, but is not limited to, S100, S100A6, S100A8, S100A9, S100A11, S100A12, S100A13, S100P, S100Z, MIP-1 alpha, TIMP-1. As used herein, the biomarker of acute phase reactants include, but is not limited to, alpha-1 antitrypsin, C-reactive protein, and fibrinogen. In preferred embodiments, the inflammatory biomarker is selected from the following thirteen biomarkers: alpha-1 antitrypsin, calcitonin, CD40, C reactive protein, EN-RAGE, erythropoietin, fibrinogen, ICAM-1, IL-6, MIP-1 alpha, myeloperoxidase, TIMP-1, and VEGF.

As used herein, the at least two biomarkers in the diagnostic biomarker panel can be any cardiomyocyte and/or inflammatory biomarker, now known or later determined, to be associated with Kawasaki Disease. The present invention further provides that the expression levels of these Kawasaki Disease diagnostic biomarkers in a biological sample from a patient are detected using a capture agent, and then compared to the reference values for the same biomarkers determined from non-Kawasaki Disease individuals. The reference values to which the detected values are compared can be those established for patients positive for Kawasaki Disease, for patients negative for Kawasaki Disease, or both. A change in the expression level of the at least two biomarkers in a biological sample from the patient relative to the reference values indicates whether the patient is or is not afflicted with Kawasaki Disease.

As used herein, the capture reagent can be any organic or inorganic chemical, biomolecule, or any fragment, homolog, analog, conjugate, or derivative thereof, that specifically interacts with the Kawasaki Disease diagnostic biomarkers. In certain embodiments, the capture reagent is a protein or antibody that specifically detects the Kawasaki Disease diagnostic biomarkers in the diagnostic biomarker panel. In yet certain embodiments, the capture reagent can be coupled to a solid support. As used herein, the biological samples from the patient are biological fluid, including, but not limited to, whole blood, plasma, serum, tears, saliva, mucous, cerebrospinal fluid, or urine.

The method disclosed in the present invention further comprises a step of detecting an expression level of a third class of biomarkers in a biological sample from the patient. As used herein, the third class of biomarkers includes, but is not limited to, an interferon type-I biomarker. In certain embodiments, the present invention provides that the patient is diagnosed as having Kawasaki Disease when the expression level of the interferon type-I biomarker is lower in the patient sample than in a sample from a control subject without Kawasaki Disease. In certain preferred embodiments, the interferon type-I biomarker-associated genes include, but are not limited to, myxovirus (influenza virus) resistance 1 (MX1), myxovirus (influenza virus) resistance 2 (MX2), interferon inducible gene (IFI2), interferon-stimulated gene (ISG15), interferon-inducible genes lymphocyte antigen 6 complex E (LY6E), oligoadenylate synthetase 1 (OAS1), oligoadenylate synthetase 2 (OAS2), oligoadenylate synthetase 3 (OAS3), interferon regulatory factor 2 (IRF2), and interferon, alpha-inducible protein 27 (IFI27).

Detecting expression levels of additional Kawasaki Disease diagnostic biomarkers, now known or later discovered to be associated with Kawasaki Disease, in the diagnostic panel, and evaluating changes in the detected expression levels of these biomarkers relative to the reference values for diagnosing a patient as having Kawasaki Disease are also within the scope of the present invention. The biomarkers described herein are well-known in the literature, as well as methods for their identification and quantification in patient samples.

It is to be understood that this invention is not limited to particular methods, reagents, compounds, compositions or biological systems, which can, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which the invention pertains. Other specifically defined terms are to be construed in a manner consistent with the definition provided herein. Although any methods and materials similar or equivalent to those described herein can be used in the practice for testing of the present invention, the preferred materials and methods are described herein. In describing and claiming the present invention, the following terminology will be used.

As used in this specification and the appended claims, the singular forms “a”, “an” and “the” include plural referents unless the content clearly dictates otherwise. Thus, for example, reference to “an antibody” includes a combination of two or more antibodies, and the like.

An “antibody” as used herein includes polyclonal and monoclonal antibodies, chimeric, single chain, and humanized antibodies, as well as antibody fragments (e.g., Fab, Fab′. F(ab⁻)₂ and Fᵥ), including the products of a Fab or other immunoglobulin expression library. With respect to antibodies, the term, “immunologically specific” or “specific” refers to antibodies that bind to one or more epitopes of a protein of interest, but which do not substantially recognize and bind other molecules in a sample containing a mixed population of antigenic biological molecules. Screening assays to determine binding specificity of an antibody are well known and routinely practiced in the art. For a comprehensive discussion of such assays, see Harlow et al. (Eds.), ANTIBODIES A LABORATORY MANUAL; Cold Spring Harbor Laboratory; Cold Spring Harbor, N.Y. (1988), Chapter 6.

Any antibody that specifically binds to the target biomarker of interest can be used in the present invention. Monoclonal and/or polyclonal antibodies can be used, from whatever source produced, as can recombinant antibodies such as single chain antibodies and phage-displayed antibodies. Antigen binding fragments of antibodies such as the Fab or Fv can also be used. Antibodies suitable for detecting Kawasaki Disease diagnostic biomarkers include monoclonal and polyclonal antibodies to any cardiomyocyte biomarkers, and any inflammatory biomarkers and any interferon type-I biomarker, such as, but not limited to, those identified herein. Antibodies can also be raised to various Kawasaki Disease biomarkers and used in the invention. Methods for raising and purifying antibodies are well known in the art. In addition, monoclonal antibodies can be prepared by any number of techniques that are known in the art, including the technique originally developed by Kohler and Milstein (1975) Nature 256:495-497.

“Capture reagent” refers to a molecule or group of molecules that specifically bind to a specific target molecule or group of target molecules. For example, a capture reagent can comprise two or more antibodies each antibody having specificity for a separate target molecule. Capture reagents can be any combination of organic or inorganic chemicals, or biomolecules, and all fragments, analogs, homologs, conjugates, and derivatives thereof that can specifically bind a target molecule.

The capture reagent can comprise a single molecule that can form a complex with multiple targets, for example, a multimeric fusion protein with multiple binding sites for different targets. The capture reagent can comprise multiple molecules each having specificity for a different target, thereby resulting in multiple capture reagent-target complexes. In certain embodiments, the capture reagent is comprised of proteins, such as antibodies.

The capture reagent can be directly labeled with a detectable moiety. For example, and not by way or limitation, an anti-NT-proBNP antibody can be directly conjugated to a detectable moiety and used in the inventive methods, devices, and kits. In the alternative, detection of the capture reagent-Kawasaki Disease biomarker complex can be by a secondary reagent that specifically binds to the biomarker or the capture reagent-biomarker complex. The secondary reagent can be any biomolecule, and is preferably an antibody. The secondary reagent is labeled with a detectable moiety. In some embodiments, the capture reagent or secondary reagent is coupled to biotin, and contacted with avidin or streptavidin having a detectable moiety tag.

Detectable moieties contemplated for use in the invention include, but are not limited to, radioisotopes, fluorescent dyes such as fluorescein, phycoerythrin, Cy-3, Cy-5, allophycoyanin, DAPI, Texas Red, rhodamine, Oregon green, Lucifer yellow, and the like, green fluorescent protein (GFP), red fluorescent protein (DsRed), Cyan Fluorescent Protein (CFP), Yellow Fluorescent Protein (YFP), Cerianthus Orange Fluorescent Protein (cOFP), alkaline phosphatase (AP), β-lactamase, chloramphenicol acetyltransferase (CAT), adenosine deaminase (ADA), aminoglycoside phosphotransferase (neo^{r}, G418^{r}) dihydrofolate reductase (DHFR), hvgromycin-B-phosphotransferase (HPH), thymidine kinase (TK), lacZ (encoding α-galactosidase), and xanthine guanine phosphoribosyltransferase (XGPRT), Beta-Glucuronidase (gus), Placental Alkaline Phosphatase (PLAP), Secreted Embryonic Alkaline Phosphatase (SEAP), or Firefly or Bacterial Luciferase (LUC). Enzyme tags are used with their cognate substrate. As with many of the standard procedures associated with the practice of the invention, skilled artisans will be aware of additional labels that can be used.

“Biomolecules” include proteins, polypeptides, nucleic acids, lipids, polysaccharides, monosaccharides, and all fragments, analogs, homologs, conjugates, and derivatives thereof. Kawasaki Disease diagnostic biomarkers can be any biomolecule that has been associated with the presence of Kawasaki Disease in a patient, including those currently known and those discovered in the future. Also contemplated are precursors and metabolites associated with such biomarkers. It is contemplated that if a microbial etiologic agent such as a bacteria, fungus, virus, or the like is later determined to be the causative agent for Kawasaki Disease, then that microbial etiologic agent can be used as a biomarker in the inventive devices and methods. The Kawasaki Disease diagnostic biomarkers for use in the invention include, but are not limited to, cardiomyocyte biomarkers and inflammatory biomarkers, including, but not limited to, a biomarker of endothelial damage, a biomarker of endothelial cell activation and leukocyte adhesion, a biomarker of macrophage monocytes, and a biomarker of acute phase reactants.

The cardiomyocyte biomarkers include, but are not limited to, N-terminal pro-B-type natriuretic peptide (NT-proBNP), C-type natriuretic peptide (CNP), atrial natriuretic peptide (ANP), high-sensitivity Troponin, IL-33, or ST2. The inflammatory biomarkers include, but are not limited to, vascular endothelial growth factor (VEGF), CD40, intercellular adhesion molecule-1 (ICAM-1), vascular cell adhesion molecule-1 (VCAM-1), carcinoembryonic antigen cell adhesion molecule-1 (CEACAM-1), EN-RAGE, pro-calcitonin, calcitonin, IL-6, IL-4, myeloperoxidase (MPO), osteoprotegrin (OPG), neutrophil elastase, MMP-8, SPARC, JAM3, SMOX, ITGB5, ITGA2B, endothelin-1, SCUBE1, S100, S100A6, S100A8, S100A9, S100A11, S100A12, S100A13, S100P, S100Z, MIP-1 alpha, TIMP-1, alpha-1 antitrypsin, C-reactive protein, and fibrinogen.

The interferon type-I biomarkers include, but are not limited to, myxovirus (influenza virus) resistance 1 (MX1), myxovirus (influenza virus) resistance 2 (MX2), interferon inducible gene (IFI2), interferon-stimulated gene (ISG15), interferon-inducible genes lymphocyte antigen 6 complex E (LY6E), oligoadenylate synthetase 1 (OAS1), oligoadenylate synthetase 2 (OAS2), oligoadenylate synthetase 3 (OAS3), interferon regulatory factor 2 (IRF2), and interferon, alpha-inducible protein 27 (IFI27).

In certain embodiments, the capture reagent is immobilized on a solid support. The solid support to which the capture reagent is coupled can be any solid support described herein. Examples of suitable solid supports include, but are not limited to, glass, plastic, metal, latex, rubber, ceramic, polymers such as polypropylene, polyvinylidene difluoride, polyethylene, polystyrene, and polyacrylamide, dextran, cellulose, nitrocellulose, Polyvinylidene Fluoride (PVDF), nylon, amylase, and the like. A solid support can be flat, concave, or convex, spherical, cylindrical, and the like, and can be particles, beads, membranes, strands, precipitates, gels, sheets, containers, wells, capillaries, films, plates, slides, and the like. The solid support can be magnetic, or a column. Sites on the solid support not coupled with the capture reagent can be blocked to prevent non-specific binding of marker molecules to the solid support. Blocking reagents and procedures are well known in the art.

The capture reagent can be immobilized on the solid support by any means suitable in the art, such as adsorption, non-covalent interactions such as hydrophobic interactions, hydrophilic interactions, van der Waals interactions, hydrogen bonding, and ionic interactions, electrostatic interactions, covalent bonds, or by use of a coupling agent. Coupling agents include glutaraldehyde, formaldehyde, hexamethylene diisocyanate, hexamethylene diisothiocyanate, N,N′-polymethylene bisiodoacetamide, N,N′-ethylene bismaleimide, ethylene glycol bissuccinimidyl succinate, bisdiazobenzidine, 1-ethyl-3-(3-dimethylaminopropyl) carbodiimide, succinimidyl 3-(2-pyridyldithio)propionate (SPDP), N-succinimidyl 4-(N-maleimidomethyl)cyclohexane-1-carboxylate (SMCC), N-sulfosuccinimidyl 4-(N-maleimidomethyl)-cyclohexane-1-carboxylate, N-succinimidyl (4-iodoacetyl)-aminobenzoate, N-succinimidyl 4-(1-maleimidophenyl)butyrate, N-(epsilon-maleimidocaproyloxy)succinimide (EMCS), iminothiolane, S-acetylmercaptosuccinic anhydride, methyl-3-(4′-dithiopyridyl) propionimidate, methyl-4-mercaptobutyrylimidate, methyl-3-mercaptopropionimidate, N-succinimidyl-S-acetylmercaptoacetate, avidin, streptavidin, biotin, Staphylococcus aureus protein A, and the like.

In certain embodiments, the solid support is a test strip. The test strips may be prepared for use with a particular reader such as a Triage® Meter (Biosite Inc., San Diego, Calif.). Any suitable reader, as will be appreciated by those of skill in the art, can be used in the present invention. In some embodiments, the test strip comprises at least two sections. The at least two sections include a section for applying and receiving a biological sample, and a section for specific capture of desired biomarkers. In other embodiments, the test strip is capable of use in a lateral flow immunoassay reader. In a lateral flow immunoassay test strip, a single fluid flow pathway is formed via membranes that provide a substratum for immunoreactions. Fluid flow can be facilitated, for example, by partially overlapping absorbent, porous, or bibulous materials placed on the strip. Fluid flow may also be facilitated using osmotic agents such as salts or sugars placed along the fluid flow pathway of the test strip.

In other certain embodiments, the biological sample or molecules separated or purified from the biological sample, are immobilized on a solid support. The biological sample can be obtained from any location in a patient in which the Kawasaki Disease markers are likely to be found. For example, a biological sample can be obtained from biological fluids such as tears, saliva, mucous, whole blood, serum, plasma, cerebrospinal fluid, urine, and the like. A biological sample could also be obtained from specific cells or tissue, or from any secretions or exudate. In certain embodiments, the biological sample is a biological fluid obtained from peripheral blood. Techniques for purification of biomolecules from samples such as cells, tissues, or biological fluid are well known in the art. The technique chosen may vary with the tissue or sample being examined, but it is well within the skill of the art to match the appropriate purification procedure with the test sample source.

The general format of the assays involve contacting the capture reagent with a biological sample containing the analytes of interest, namely the Kawasaki Disease diagnostic biomarkers, which may be distinguished from other components found in the sample. Following interaction of the analyte with the capture reagent, the system can be washed and then directly detected or detected by means of a secondary reagent as exemplified herein.

A variety of assay formats can be used to carry out the inventive methods. Immunoassays are preferred and include, but are not limited to, ELISA, radioimmunoassays, competition assays, bead agglutination assays, lateral flow immunoassays, immunochromatographic test strips, dipsticks, migratory format immunoassays, and the like. Other suitable immunoassays will be known to those of relevant skill in the art. Microscopy can also be used.

As the various Kawasaki Disease biomarkers identified to date can be present at detectable levels within normal subjects (those without Kawasaki Disease), it may be necessary to quantitatively measure the levels of each biomarker being analyzed in the diagnostic assay. In such cases, modulation of expression levels of the biomarkers relative to standards/controls and/or reference values will be indicative of the presence or absence of Kawasaki Disease. Normal expression levels, i.e., those of healthy non-Kawasaki Disease subjects, of the various markers can be empirically determined according to any of various techniques that are known in the art. The normal expression levels can serve as a reference value against which the expression levels in suspected Kawasaki Disease patients can be compared. Significant deviation (positive or negative) over expected normal expression levels of the at least two Kawasaki Disease diagnostic biomarkers is indicative of the presence of the disease in the patient. Similarly, lack of significant deviation over expected normal expression levels of at least two Kawasaki Disease diagnostic biomarkers can be indicative of the absence of disease in the patient.

The expression levels observed in confirmed Kawasaki Disease patients can also serve as a standard against which the expression levels in suspected Kawasaki Disease patients can be compared. Similar levels of expression of at least two Kawasaki Disease diagnostic biomarkers between the known patient and suspected patient is indicative of the presence of the disease in the patient. In such cases, it is expected that the biomarker expression level in both the known and suspected samples will significantly deviate from the level of expression present in healthy (no disease) subjects.

The present invention further provides a kit for diagnosing Kawasaki Disease comprising a capture reagent comprising a) one or more detectors specific for at least two Kawasaki Disease diagnostic biomarkers, wherein the first biomarker is a cardiomyocyte biomarker, and the second biomarker is an inflammatory biomarker, b) a detection reagent, and c) instructions for using the kit to diagnose a patient as having Kawasaki Disease when the expression levels of the at least two diagnostic biomarkers in a patient test sample are higher than the expression levels of the same biomarkers in a control subject without Kawasaki Disease. In certain embodiments, the cardiomyocyte biomarker includes, but is not limited to, N-terminal pro-B-type natriuretic peptide (NT-proBNP), C-type natriuretic peptide (CNP), atrial natriuretic peptide (ANP), high-sensitivity Troponin, IL-33, or ST2, and the inflammatory biomarker includes, but is not limited to, a biomarker of endothelial cell, platelet, and leukocyte damage, activation and adhesion, a biomarker of macrophage monocytes, and a biomarker of acute phase reactants, as discussed above.

The present invention further provides a kit that comprises a capture reagent comprising a detector that specifically detects an interferon type-I biomarker, and instructions for using the kit to diagnose a patient as having Kawasaki Disease when the expression level of the interferon type-I biomarker is lower in patient biological sample than in a control subject without Kawasaki Disease. A kit comprising other capture reagents that comprise detectors specifically detect other additional Kawasaki Disease diagnostic biomarkers now know or later discovered, and instructions to diagnose a patient as having Kawasaki Disease when the expression levels of these diagnostic biomarkers in patient are different from the expression levels of the same biomarkers in a control subject without Kawasaki Disease is also within the scope of the present invention.

The kits of the present invention can further comprise appropriate positive and negative controls against which a biological sample from a patient can be compared. The kits can further comprise reference values established for the expression of Kawasaki Disease patients positive for Kawasaki Disease, for patients negative for Kawasaki Disease, or both. In kits not having a detection reagent, the capture reagent is coupled to a detectable moiety, or biotin, as described herein.

In some embodiments, the kits further include a solid support to immobilize the capture reagent. The solid support can be any solid support described herein. The kit may further include coupling agents to facilitate immobilization of the capture reagent to the solid support. In some embodiments, the capture reagent is provided coupled to the solid support.

The kits can also include positive and negative controls. Positive controls can be a supply of Kawasaki Disease diagnostic biomarkers specifically recognized by the capture reagent. Such biomarkers are at a concentration indicative of the presence of Kawasaki Disease. Whole blood, plasma, or serum from an individual known to have Kawasaki Disease can also serve as a positive control. Negative controls can be any molecules not associated with Kawasaki Disease. Blood or serum from an individual known to not have Kawasaki Disease can also serve as a negative control. The kits may also include reference values for expression levels of Kawasaki Disease associated markers in healthy subjects and/or confirmed Kawasaki Disease patients. The kits can contain materials sufficient for one assay, or can contain sufficient materials for multiple assays.

Also featured are devices to diagnose Kawasaki Disease. The Kawasaki Disease diagnostic device disclosed in the present invention comprises a) a capture reagent comprising one or more detectors specific for at least two Kawasaki Disease diagnostic biomarkers, wherein the first biomarker is a cardiomyocyte biomarker, and the second biomarker is an inflammatory biomarker, and b) detecting reagents for detecting an expression level of the at least two diagnostic biomarkers in a biological sample from the patient, wherein the patient is diagnosed as having Kawasaki Disease when the expression levels of the at least two diagnostic biomarkers in the patient biological sample are higher than the expression levels in a control subject without Kawasaki Disease. The capture reagent of the devices can be an organic or inorganic chemical, biomolecule, or any fragment, homolog, analog, conjugate, or derivative thereof that specifically interacts with the Kawasaki Disease diagnostic biomarkers. In certain embodiments, the capture reagent is a protein and/or an antibody, and may be immobilized on a solid support.

The device of the present invention also comprises a) a capture reagent comprising a detector specific for an interferon type-I biomarker, and b) detecting reagents for detecting an expression level of the interferon type-I biomarker, wherein the patient is diagnosed as having Kawasaki Disease when the expression level of the interferon type-I biomarker is lower in patient biological sample than in a control subject without Kawasaki Disease. The device of the present invention can also comprise a capture reagent comprising one or more detector for other interferon Kawasaki Disease diagnostic biomarkers, now known or later discovered, and detecting reagents for detecting expression levels of these Kawasaki Disease diagnostic biomarkers in a biological sample from a patient, wherein the patient is diagnosed as having Kawasaki Disease when the expression levels of these diagnostic biomarkers in patient biological sample are different from the expression levels of the same biomarkers in a control subject without Kawasaki Disease.

The present invention further provides a methodology for determining an algorithm to weight the biomarker data for optimum Kawasaki Disease diagnosis. In certain embodiments, multivariable logistic regression analysis can be used to identify independent predictors for diagnosing Kawasaki Disease or predicting IVIG-resistance. An optimal cut-point is selected by receiver-operator characteristic (ROC) curves for proteins that are significant by logistic regression. In an iterative process, independent predictors are then combined using Classification and Regression Tree (CART) analysis to create a group of biomarkers that diagnose Kawasaki Disease. Validation of these statistical models developed on the “training” cohort is conducted by selecting (at random) independent “testing” cohorts of Kawasaki Disease and control patients from the pool of patients in the study database. Further, re-sampling techniques such as k-fold cross-validation and permutation tests are particularly useful in taming complicated inferential problems that arise, such as obtaining unbiased estimates of the error rate of a prediction rule or constructing significance tests of nominally correct size when the usual distribution theory is inadequate.

The ROC curves identify the cut-points for each value. Then the CART analysis provides a decision tree analysis that tests different “paths” through the tests. For example, one can first look at NT-pro-BNP. If the patient value is >x, then look at Biomarker A. If Biomarker A is >y, then the diagnosis is Kawasaki Disease. If Biomarker A is <y, look at Biomarker B, C, D, or more, until at least one biomarker is >y. Then the computer tests these algorithms on the data set generated and keeps refining the “paths” through the tests until the most robust combination is found. Therefore, the invention provides all the data that needed to generate particular algorithms for how these test can be used in a panel to diagnose Kawasaki Disease.

Other embodiments and uses are apparent to one skilled in the art in light of the present disclosures. Those skilled in the art will appreciate that numerous changes and modifications can be made to the embodiments of the invention and that such changes and modifications can be made without departing from the spirit of the invention. It is, therefore, intended that the appended claims cover all such equivalent variations as fall within the true spirit and scope of the invention.

The following example is provided to describe the invention in more detail. It is intended to illustrate, not to limit the invention.

### Example 1

### NT-proBNP and ST2 as Indicators of Myocardial Strain in Acute Kawasaki Disease (KD)

### Study Population and Sample Collection

KD patients (n=96), febrile controls (n=93), and healthy pediatric controls (n=50) were enrolled at Rady Children's Hospital, San Diego, Calif. after parental informed consent. The human subjects protocol was reviewed and approved by the Institutional Review Board at UCSD. All KD patients had fever and ≧4 of the 5 principal clinical criteria for KD (rash, conjunctival injection, cervical lymphadenopathy, changes in the oral mucosa, and changes in the extremities) or 3 criteria plus coronary artery abnormalities documented by echocardiography (Liu et al (2006) Curr Opin Microbiol. 9:312-9). All febrile control patients had nasopharyngeal and stool viral cultures. Controls classified as having acute adenoviral infection (n=14) had fever for at least 3 days, a negative throat culture for Group A β-hemolytic streptococcus (GABHS), and a positive nasopharyngeal (NP) culture for adenovirus. Controls classified as having acute streptococcal scarlet fever (n=3) had fever, a diffuse scarlitiniform rash, clear conjunctivae, negative NP and stool viral cultures, and a positive rapid test for GABHS. Control subjects with bacterial infections included 2 with cellulitis, 2 with scalded skin syndrome, and 3 with streptococcal pharyngitis. Controls whose fever and systemic signs were attributed to a non-cultivable virus (viral syndrome, n=56) had a negative throat culture for GABHS, negative NP and stool viral cultures, and a clinical illness that resolved without specific therapy. Healthy pediatric control patients (n=50) were children less than 6 years of age undergoing minor elective surgery for polydactyly.

Clinical data including sex, ethnicity, race, age, day of illness (first day of fever=illness Day 1), results of clinical laboratory testing, response to IVIG therapy, and coronary artery status were recorded for all KD subjects. IVIG resistance was defined as persistent or recrudescent fever (T≧100.4° F. rectally or orally) 36 h following completion of the IVIG infusion (2 g/kg). The internal diameters of the right coronary and left anterior descending arteries were classified by echocardiography as normal (<2.5 standard deviations [z score] from the mean, normalized for body surface area (Popper et al. (2007) Genome Biol. 8: R261), dilated (2.5≦z score<4.0), or aneurysmal (saccular or fusiform dilatation of a coronary artery segment with z score ≧4.0). Blood was obtained for plasma biomarkers and determination of complete blood count and differential, C-reactive protein (CRP), and erythrocyte sedimentation rate (ESR) at two timepoints: acute (Illness Day 3-10 and prior to IVIG administration) and convalescent (Illness Day ≧21).

### Biomarker Measurements

Plasma (EDTA) NT-proBNP concentrations were determined for the following subjects: 27 acute KD, 4 convalescent KD, 41 febrile controls, and 20 healthy control. Plasma (Na+ citrate) ST2 concentrations were determined for 12 acute KD, 47 convalescent KD, 40 febrile controls, and 30 healthy controls. Of these subjects, 57 acute KD subjects, 8 convalescent KD subjects, and 12 febrile controls had both NT-proBNP and ST2 plasma levels determined.

NT-proBNP concentration was measured using a biotin-coupled anti-NT-proBNP antibody/streptavidin solid-phase chromatographic immunoassay (StatusFirst CHF NT-proBNP test devices, Nanogen, San Diego, Calif.), in combination with the DXpress Reader (Nanogen, San Diego, Calif.). ST2 levels were determined using the Presage ST2 assay kit (Critical Diagnostics, San Diego, Calif.).

### Statistical Analysis

Data were analyzed using STATA (Stata Corp., College Station, Tex.) and GraphPad Prism (GraphPad Software. Inc., La Jolla, Calif.) software, and presented as the median and interquartile range. Comparisons of plasma NT-proBNP and ST2 levels between KD and control groups were evaluated with the Kruskal-Wallis test, followed by the Dunn's post hoc test. The Mann-Whitney U test was used to compare clinical data between acute KD and febrile control subjects. Correlation between variables was evaluated using Spearman's rank sum test. Relationship between KD biomarker levels and categorical data (IVIG response and CA status) was analyzed with Pearson's chi-square test. For all tests, p<0.05 was considered significant.

### Results

### Patient Characteristics

The demographic and clinical characteristics of the KD and control subjects are shown in Table 1.

TABLE 1
Clinical and laboratory characteristics of subjects in whom plasma
NT-proBNP and ST2 concentration were determined

| | NT-proBNP (pg/ml) | | ST2 (U/ml) | |
|---|---|---|---|---|
| | Acute KD | FC | Acute KD | FC |
| Characteristics | (n = 84) | (n = 53) | (n = 69) | (n = 52) |
| Age (y) | 3.16 | 3.46 | 3.33 | 3.40 |
| | [0.25-15.11] | [0.15-16.63] | (0.17-11.06) | (0.15-16.69) |
| Male, n (%) | 53 | 31 | 43 | 29 |
| | (63.1) | (58.5) | (62.3) | (55.8) |
| Aneurysm, | 9 | NA | 6 | NA |
| n (%) | (10.7) | | (8.7) | |
| IVIG resistant, | 24 | NA | 17 | NA |
| n (%) | (28.6) | | (24.6) | |
| CRP (mg/dl) | 7.7 | 3.1 | 7.3 | 1.6 |
| | [4.8-16.25] | [1-5.9]* | (4.0-16.8) | (0.6-4.1)§ |
| ESR (mm/h) | 59.0 | 32.0 | 56.0 | 20 |
| | [39.0-75.3] | [18.5-43.5]† | (39.3-75.0) | (10-35)∥ |
| WBC | 13.4 | 9.2 | 13.1 | 7.4 |
| (×10³/mm³) | [10.9-16.78] | [6.8-13.9]‡ | (10.8-18.4) | (5.2-11.4)¶ |

Values are represented as median [interquartile range] due to non-Gaussian distributions.
*35/53 febrile control subjects had information on CRP;
†35/53 febrile subjects had information on ESR;
‡45/53 febrile subjects had information on WBC;
§38/52 febrile subjects had information on CRP;
∥48/52 febrile subjects had information on ESR;
¶50/52 febrile subjects had information on WBC.
CRP = C-reactive protein;
ESR = erythrocyte sedimentation rate;
FC = febrile control;
IVIG = intravenous immunoglobulin;
NA = not applicable.

### Plasma NT-proBNP and ST2 Levels

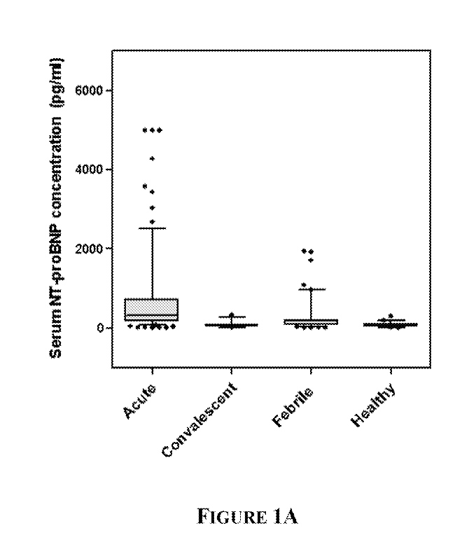

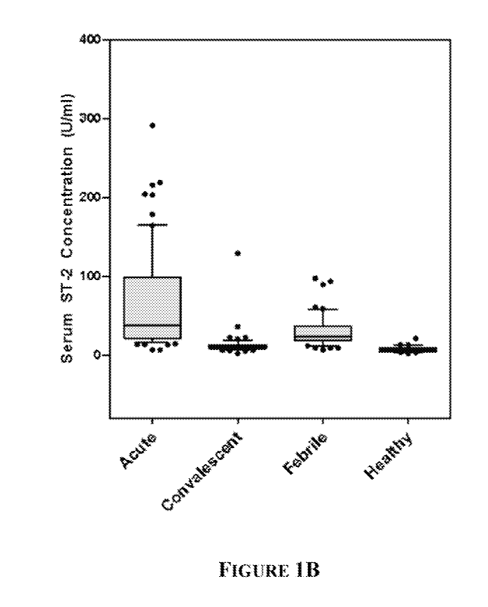

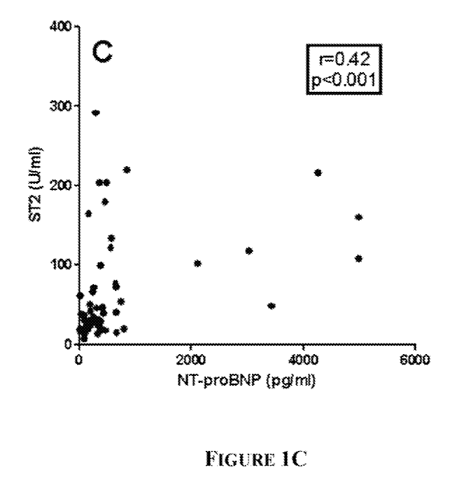

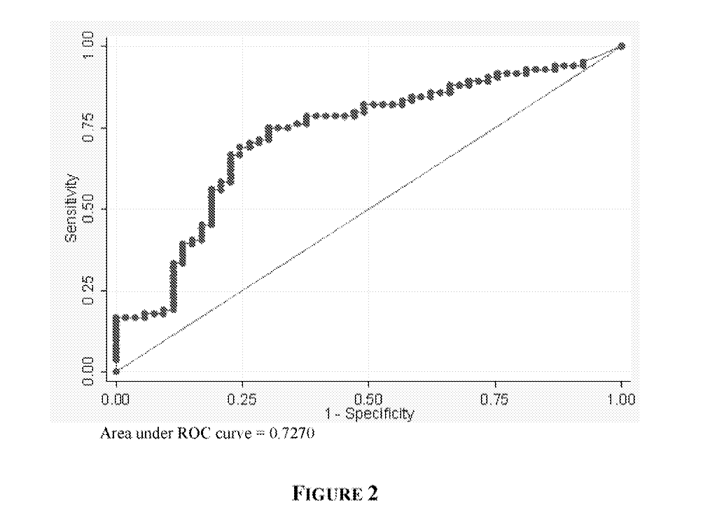

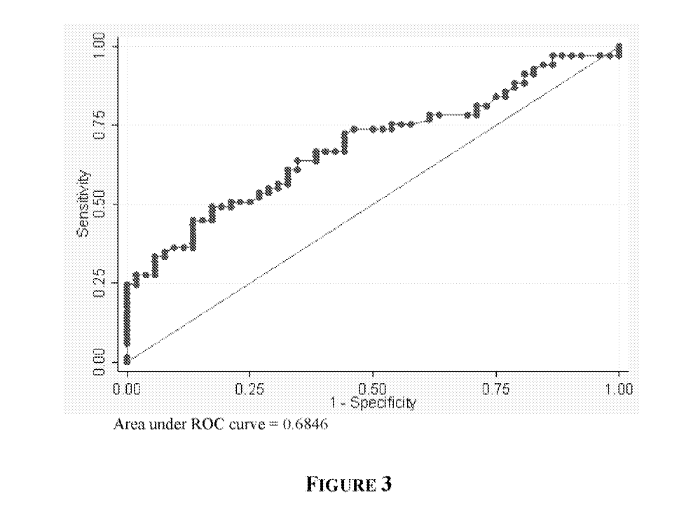

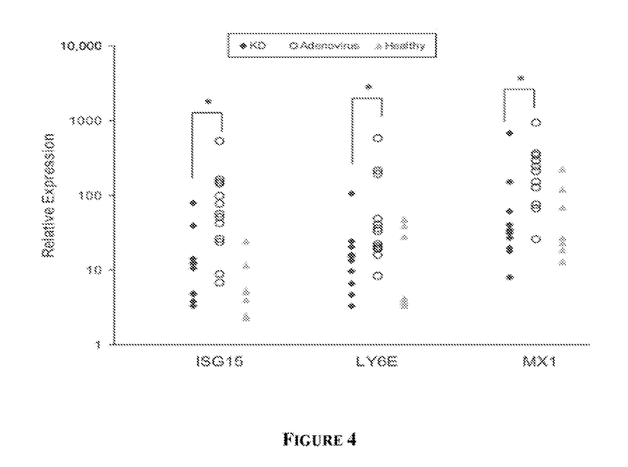

Plasma NT-proBNP (FIG. 1A) and ST2 (FIG. 1B) levels were significantly different among KD and control groups (p<0.00001, Kruskal-Wallis test). Acute KD subjects had higher NT-proBNP levels (327.6 [165.7-728.2] pg/ml) than convalescent KD (56.1 [31.1-84.1] pg/ml; p<0.05, Dunn's test), febrile controls (111.2 [75.3-197.4] pg/ml; p<0.05), and healthy controls (65.2 [45.0-112.3] pg/ml; p<0.05). NT-proBNP values were not significantly different among convalescent KD, febrile control, and healthy controls. ST2 concentration was significantly elevated in acute KD (37.4 [21.8-98.4] U/ml), in comparison to convalescent KD (10.6 [8.3-13.4] U/ml; p<0.05) and healthy control subjects (7.2 [4.3-9.7] U/ml; p<0.05). In 57 acute KD patients for whom both NT-proBNP and ST2 were determined, a positive relationship was noted (FIG. 1C) (r=0.42, p<0.001).

No significant correlations were observed between NT-proBNP and ST2 concentrations and ESR or WBC (Table 2). NT-proBNP and ST2 concentrations were weakly correlated with CRP (r=0.435; p<0.001, and r=0.412; p<0.001, respectively) and Illness day (r=−0.45; p<0.001, and r=p<0.001 respectively) (Table 2).

TABLE 2
Correlation of biomarkers with clinical and laboratory parameters

| | | |
|---|---|---|
| | NT-proBNP | NT-proBNP |
| | (pg/ml), | (pg/ml), |
| e | n = 84 | n = 84 |

| Covariates | r | p-Value | r | p-Value |
|---|---|---|---|---|
| CRP (mg/dl) | 0.435 | <0.001 | 0.412 | <0.001 |
| ESR (mm/h) | −0.014 | 0.903 | 0.01 | 0.422 |
| WBC (×10³/mm³) | −0.023 | 0.835 | 0.143 | 0.241 |
| Illness day | −0.46 | <0.001 | −0.45 | <0.001 |

CRP = C-reactive protein;
ESR = erythrocyte sedimentation rate;
NT-proBNP = N-terminal propeptide B-type natriuretic peptide;
ST2 = interleukin-1 receptor family member;
WBC = white blood cell count.

No significant differences were noted between biomarker levels and IVIG response treatment (p=0.023) or coronary artery outcome (p=0.015) (Table 3, Table 4).

TABLE 3
Relationship between biomarker levels and IVIG response

| | Treatment | | | | |
|---|---|---|---|---|---|
| | response | n | Median | 95% CI | p-Value |
| NT-proBNP | Resistant | 24 | 454 | 358-854 | |
| (n = 84)* | Responder | 54 | 296 | 201-408 | |
| ST2 (n = 69)† | Resistant | 17 | 51 | 29-203 | |
| | Responder | 47 | 37 | 28-53 | |

Plasma NT-proBNP and ST2 concentration in acute KD subjects were compared with their IVIG treatment response.
Acute KD subjects were categorized as being resistant or responsive to IVIG infusion.
*Six out of 84 patients received late IVIG treatment (≧11 days); hence, were not included in this study.
†Five out of 69 patients received late IVIG treatment (≧11 days); hence, were not included in this study.
NT-proBNP = N-terminal propeptide B-type natriuretic peptide;
ST2 = interleukin 1 receptor family.

TABLE 4
Comparison of coronary artery status with NT-proBNP and ST-2 levels

| | Treatment | | | | |
|---|---|---|---|---|---|
| | response | n | Median | Range | P-value |
| NT-proBNP | Normal | 55 | 300 | 187-358 | |
| (n = 84) | Aneurysm + | 29 | 577 | 252-854 | |
| | dilation | | | | |
| ST2 (n = 69) | Normal | 47 | 36 | 28-48 | |
| | Aneurysm + | 22 | 56 | 25-107 | |
| | dilation | | | | |

Comparison of coronary artery (CA) status with plasma NT-proBNP and ST2 measurements of acute KD subjects.
CA status was either categorized as “normal” or “aneurysm and dilation.”
NT-proBNP = N-terminal propeptide B-type natriuretic peptide;
ST2 = interleukin 1 receptor family.

### Example 2

### Transcript Abundance Profiles Distinguish Kawasaki Disease from Other Rash/Fever Illnesses

In this study, patterns of whole blood gene expression in acute KD patients, and patients with illnesses of similar clinical presentation and well-defined etiology were compared. Patterns of gene expression and corresponding biological programs that differed between KD and the other illnesses were identified, which were able to distinguish between KD and adenovirus infection on the basis of gene expression patterns. The results from this study can be exploited to devise a diagnostic test for KD that may lead to more accurate and timely diagnosis of these patients.

To identify unique features of KD, gene expression patterns in the blood of 23 children with acute KD and 18 age-matched febrile controls were compared. Genes associated with platelet and neutrophil activation were expressed at higher levels in KD patients than patients with adenovirus infections or systemic toxic drug reactions but not scarlet fever; an expression program associated with B cell activation was also expressed at higher levels in the KD patients than the controls. The most striking finding was the absence of interferon-stimulated gene expression in the KD patients; this was confirmed by RT-PCR in an independent cohort of KD subjects. This study successfully predicted 21 of 23 KD patients and 6 of 7 Adenovirus patients using 10-fold cross validation and a set of 38 genes. These findings provide insight into the molecular features that distinguish KD from other febrile illnesses, and support the feasibility of developing novel diagnostic reagents for KD based on the host response.

### Materials and Methods

### Study Population and Sample Collection

KD patients (n=23), febrile controls (n=18), and healthy pediatric controls (n=10) were enrolled at two clinical sites (Rady Children's Hospital, San Diego, Calif. and Children's Hospital Boston, Mass.) after parental informed consent. The human subjects protocol was reviewed and approved by the Institutional Review Boards at UCSD, Children's Hospital Boston, and Stanford University. All KD patients had fever and >4 of the 5 principal clinical criteria for KD (rash, conjunctival injection, cervical lymphadenopathy, changes in the oral mucosa, and changes in the extremities) or 3 criteria plus coronary artery abnormalities documented by echocardiography. All febrile control patients had nasopharyngeal and stool viral cultures. Controls classified as having acute adenoviral infection had fever for at least 3 days, a negative throat culture for Group A β-hemolytic streptococcus (GABHS), and a positive nasopharyngeal (NP) culture for adenovirus. Controls classified as having acute streptococcal scarlet fever had fever, a diffuse scarlitiniform rash, clear conjunctivae, negative NP and stool viral cultures, and a positive rapid test for GABHS. Controls whose fever and systemic signs were attributed to a drug reaction had a negative throat culture for GABHS, negative NP and stool viral cultures, and a history of a drug known to cause hypersensitivity reactions. Healthy pediatric control patients (n=10) were children less than 6 years of age undergoing minor elective surgery for polydactyly.

Clinical data including sex, ethnicity, race, age, day of illness (first day of fever=illness Day 1), results of laboratory testing, response to IVIG therapy, and coronary artery status were recorded for all subjects. IVIG non-response was defined as persistent or recrudescent fever (T≧100.4° F. rectally or orally) 48 h following completion of the IVIG infusion (2 g/kg). The internal diameters of the right coronary and left anterior descending arteries were classified by echocardiography as normal (<2.5 standard deviations [z score] from the mean, normalized for body surface area), dilated (2.5≦z score<4.0), or aneurysmal (saccular or fusiform dilatation of a coronary artery segment with z score ≧4.0).

Blood was obtained for determination of complete blood count and differential, C-reactive protein (CRP), erythrocyte sedimentation rate (ESR), alanine amino transferase (ALT), and gamma glutamyltransferase (GGT), and for RNA studies (2.5 ml in PAXgene tube (Qiagen)). PAXgene tubes were stored at 4° C. for no more than 5 days and RNA was purified according to the manufacturer's instructions.

### DNA Microarray Hybridization

RNA transcripts in the samples and a standard reference RNA (Universal Human Reference RNA, Stratagene, La Jolla, Calif.) were amplified using the MessageAmp aRNA amplification kit (Ambion, Austin, Tex.). Sample and reference transcripts were then reverse-transcribed, labeled with fluorescent dyes (Cy5 and Cy3, respectively), mixed together, and hybridized to cDNA microarrays as previously described. The arrays used for these studies contain 37,632 spots derived from cDNA clones representing ˜18,000 unique human genes (Alizadeh et al., Nature 2000; 403:503-11). Images of hybridized arrays were obtained using a Genepix 4000B microarray scanner, and analyzed with Genepix 5.0 (Axon Instruments, Union City, Calif.).

### Microarray Data Filtering and Analysis

Data were filtered to include only clones that met the following criteria for at least 80% of the samples tested: signal intensity 2.5-fold above background in either the Cy5 (sample) or Cy3 (reference) channel, and a regression correlation for the two channels of at least 0.6 across each measured element. A normalization factor was applied so that the mean log2 ratio for each array (sample) was zero, and data for each clone were then median-centered across all observations. Selected data were organized using a hierarchical clustering algorithm based on a Pearson correlation metric, with average linkage clustering, and visualized using Treeview. GeneTrail, the Molecular Signatures Database, and NextBio were used to identify Gene Ontology (GO) terms, biological pathways from the Kyoto Encyclopedia of Genes and Genomes (KEGG), and other array datasets associated with gene sets in our analysis. Terms in GeneTrail were identified using a false discovery rate (FDR) of 5%, and minimum membership of 3 genes. Significance Analysis of Microarrays (SAM) was used to identify genes significantly associated with differences between KD patients and each of the three groups of controls. Prediction Analysis of Microarrays (PAM) with 10-fold cross-validation was used to identify and assess the predictive values of genes distinguishing KD and adenovirus patients. Potential differences in clinical and laboratory parameters were tested using STATA v.7 (STATA, College Station, Tex.).

### Quantitative RT-PCR

Levels of ISG15, LY6E, and MX1 mRNA were measured using TaqMan 5′-nuclease Gene Expression Assays (Applied Biosystems, Foster City, Calif., USA). cDNA was synthesized using Oligo(dT)20 (Invitrogen, Carlsbad, Calif.) and Superscript III RT (Invitrogen, Carlsbad, Calif.), in accordance with the manufacturer's instructions. PCR reactions were prepared using the TaqMan Universal PCR Master Mix (Applied Biosystems, Foster City, Calif., USA), and cDNA derived from 20 ng of total RNA. Relative abundance of the target transcripts was calculated by comparison to a standard curve, and normalized to the expression level of TATA box binding protein-Associated Factor, RNA polymerase I, B (TAF1B). ABI assay catalog numbers are as follows: ISG15: Applied Biosystems (ABI) cat. # Hs00192713_ml; LY6E: ABI cat #Hs00158942_ml; MX1: ABI cat# Hs00182073_ml; TAF1B: ABI cat. # Hs00374547_ml.

### Results

Transcript Profiles of KD Patients Differ from Patients with Adenovirus Infections or Drug Reactions

Whole blood RNA samples from 23 children with KD, and from 18 children with a well-defined illness and similar clinical presentation, were processed and hybridized to cDNA arrays; clinical characteristics of these patient groups are presented in Table 5.

TABLE 5
Clinical Characteristics of Study Patients with Kawasaki Disease (KD) and Febrile Control Subjects

| | | Control subjects, by illness | | |
|---|---|---|---|---|
| | Patients with | Adenovirus | Group A Streptococcus | |
| | KD | infection | infection | Drug reaction |
| Characteristic | (n = 23) | (n = 8) | (n = 5) | (n = 5) |
| Age at illness onset, | 2.5 | 2.6 | 3.8 | 8.6 |
| years | (0.4-12.8) | (0.5-8.1) | (1.9-6.8) | (0.9-15.9) |
| Sex | 15 male, 8 | 5 male, 3 | 3 male, 2 female | 5 male |
| | female | female | | |
| Day of illness [symbol] | 6 | 5.5 | 3 | 6 |
| | (4-12) | (3-11) | (2-21)^{b} | (4-14) |
| CA status | 13 N, 4 A, 6 D | NA | NA | NA |
| IVIG response | 18 R, 3 NR, | NA | NA | NA |
| | 2 LT | | | |
| White blood cell count, | 13.2 | 14.5 | 13.6 | 5.7 |
| ×10³/mm³ | (6.5-23.5) | (6-22.5) | (6.8-14.8) | (3.2-15.3)^{b} |
| Neutrophils, % | 49 | 50 | 60 | 47 |
| | (12-69) | (29-74) | (20-74) | (21-72) |
| Bands, % | 12 | 8 | 8 | 9 |
| | (0-53) | (0-26) | (5-66) | (4-15) |
| Absolute neutrophil | 8.6 | 6.6 | 11.0 | 2.8 |
| count, ×10³/mm³ | (2.8-19.0) | (4.0-16.9) | (4.5-12.1) | (1.8-4.3)^{b} |
| Monocytes, % | 5 | 11 | 6 | 5 |
| | (0-11) | (2-15)^{b} | (3-13) | (4.6-6.0) |
| Eosinophils, % | 3 | 0 | 1 | 4 |
| | (0-11) | (0-1)^{b} | (0-11) | (0-22) |
| Lymphocytes, % | 27 | 19 | 11 | 37 |
| | (8-53) | (13-65) | (5-25)^{b} | (17-43) |
| zHg | 1.0 | −0.4 | −1.0 | 1.0 |
| | (−3.5 to 3.8) | (−1.6 to 0.0) | (−2.5 to 0.7) | (−1.1 to 2.7)^{b} |
| Hematocrit, % | 32.7 | 33.6 | 35.2 | 39.9 |
| | (28.9-42.7) | (32.7-37.2) | (30.2-38.8) | (36.5-45.7)^{b} |
| ESR, mm/h | 74 | 57.5 | 5 | 10 |
| | (23-140) | (13-88) | | (4-16)^{b} |
| CRP, mg/dL | 8.3 | 3.3 | 2.4 | 5 |
| | (0.8-36.9) | (2.0-7.8)^{b} | | (1.4-7.5) |
| Platelet count, ×10³/mm³ | 443 | 304 | 298 | 300 |
| | (217-595) | (174-455)^{b} | (209-376)^{b} | (148-586) |
| ALT, IU/L | 52 | 13 | ND | 15 |
| | (3-550) | (0.3-66)^{b} | | (6-31)^{b} |
| GGT, IU/L | 75 | 19 | ND | 34.5 |
| | (17-233) | (12-44)^{b} | | (27-50) |

Data are medians (ranges), unless otherwise indicated A, aneurysm;
ALT, alanine aminotransferase;
CA, coronary artery;
CRP, C- reactive protein;
D, dilated;
ESR, erythrocyte sedimentation rate;
GGT, γ- glutamyl transferase;
IVIG, intravenous immunoglobulin;
LT, treated after day 10;
N, normal;
NA, not applicable;
ND, not done;
NR, IVIG nonresponder;
R, IVIG responder;
zHg, z scores for hemoglobin level, normalized by age.
^{a}Day on which samples were obtained.
^{b} [symbol] for comparison with KD group (rank sum test). [symbol]
indicates data missing or illegible when filed

To focus on the transcripts with the greatest differences among the patients, 808 transcripts were identified that varied at least three-fold from the median in at least 2 of the 41 arrays, and used unsupervised clustering to organize the samples. Sample cluster A, forming one of the two main branches of the dendrogram, consisted primarily of KD samples; 14 of the 19 samples were from KD patients, and 4 of the remaining five were from patients with scarlet fever. Sample cluster B contained 7 of 8 Adenovirus samples, along with three samples from patients with toxic drug reactions and one KD patient. The grouping of samples suggested that disease-specific transcription patterns were a prominent feature of the overall transcription profiles of each patient.

### Identification of KD-Associated Transcript Patterns

Significance Analysis of Microarrays (SAM) was used to identify specific transcripts significantly more or less abundant in the children with KD than each of the three control groups. There were 130 transcripts differentially expressed when compared with the adenovirus patients, 135 when compared to those with systemic drug reactions, and 4 when compared to those with scarlet fever; 29 transcripts were common to the first two comparisons. To identify additional transcripts whose abundance was similar across control groups but different from the KD group, transcript levels in the KD patients were compared with the transcript levels in the pooled control groups; 31 additional transcripts were identified in this manner.

To identify functional relationships associated with these overlapping lists, the set of 271 differentially expressed transcripts were clustered and applied several complementary approaches: Gene Ontology (GO) terms and annotated biological pathways were identified that were over-represented; other array datasets were also identified that were enriched for genes present in each of these four gene clusters, and information about the temporal pattern of expression of these genes in KD were incorporated.

Genes Associated with Cell Adhesion and Innate Immune Responses are Expressed at High Levels in KD Patients

Transcripts with significant differences in abundance were organized using hierarchical clustering. There were four main gene clusters among the KD-associated transcripts, each with at least 20 cDNA clones and an average Pearson correlation coefficient of 0.5. The first of these clusters (C1), consisted of 45 transcripts expressed at higher levels in KD patients than control patients. Forty-two transcripts were significantly less abundant in adenovirus patients than in children with KD, and 13 were less abundant in those with drug reactions. Genes represented in this cluster were enriched for the GO term contractile fibers (p<0.01), and included vinculin (VCL) and the myosin light chain 4 and 9 subunits (MYL4, MYL9). Each of these genes is involved in modulating cell adhesion, morphology, and migration—as are a number of other genes in this cluster, including SPARC, ITGA2B, and ITGB5, TUBB and TBCC. SPARC, SELP, ITGA2B and, TUBB1, are also known to be highly expressed in platelets; this gene set was significantly enriched for transcripts expressed during in vitro differentiation of stem cells into megakaryocytes (p<0.01) and for genes associated with hematopoietic cell maturation (p<10−5).

Cluster 2 (C2) also contained transcripts more abundant in KD patients than those with Adenovirus infections or drug reactions. Approximately half of the cDNA clones (54 of 100) were among those that were previously identified as defining the acute phase of KD in a study of temporal patterns of gene expression in KD. These included a number of antimicrobial peptides, such as bactericidal permeability-increasing protein (BPI), secretory leukocyte proteinase inhibitor (SLPI), defensin alpha 1 (DEFA1), peptidase inhibitor 3 (PI3), and other genes associated with early innate immune responses, such as S100 genes S100P and S100A12, MMP9, sortilin-like receptor 1 and Pre-B cell colony-enhancing factor (PBEF). No GO terms met the criteria for significance, but two overlapping KEGG pathways, apoptosis and insulin signaling were identified, that were significantly associated with this gene cluster (p<0.01). Genes associated with these GO terms encoded proteins in signaling pathways associated linked to immune responses and inflammation, including the interleukin 1 receptor type 1 (IL1R1) and IL1 receptor associated protein interleukin 1 receptor accessory protein (IL1RAP), inhibitor of kappaB kinase gamma (IKBKG), Phosphatidylinositol 3-kinase (PIK3R1), and regulatory type I alpha subunit (PRKAR1A). Additional cytokine receptors (IL4 receptor (IL4R) and interferon gamma receptor subunit 1 (IFNGR1)) were also present in this gene cluster. This gene set also included many genes expressed at higher levels in neutrophils compared to other leukocyte subsets (Jeffrey et at (2006) Nat. Immunol. 7:274-83), as well as genes associated with differentiation of both CD34+ myeloid cells and monocytes (p<10-14 and p<10-4, respectively).

### A B Cell Activation Program is Stably Expressed at High Levels in Some KD Patients

The 33 transcripts in cluster 3 (C3) were also significantly more abundant in KD patients than control groups. Twenty-nine were significantly different only when the pooled set of febrile controls was compared to KD, suggesting a consistent but less dramatic difference between KD patients and the febrile controls compared to clusters 1 and 2. This impression was reinforced by a sample size estimate which indicated that similar sample sizes would be needed to identify significant differences for each control group.

This set of genes was highly enriched for genes associated with B cells. The pattern of transcript abundance was associated with a gene expression program that more specifically characterizes a non-plasma cell stage of activation: transcription factor PAX5, which plays a central role in the early activation and differentiation of B cells and whose expression must be repressed for development of plasma cells, was among the group of genes more highly expressed in KD patients, as were a number of genes known to be direct targets of PAX5 transcriptional activation (e.g., CD79A, membrane-spanning 4-domains, subfamily A, member 2 (MS4A2), immunoglobulin heavy constant mu (IGHM), Spi-B transcription factor (SPiB), human leukocyte antigen (HLA)-DNA, tumor necrosis factor receptor associated factor 5 (TRAF5)). Unlike genes in clusters 1 and 2, expression of genes in cluster 3 did not diminish with the end of the acute phase of the disease; the average transcript abundance for individual KD patients was very similar weeks to months later, during the convalescent phase of the disease (p=0.92, paired t-test).

Interferon Responsive Genes are Expressed at Lower Levels in KD Patients than Adenovirus Patients

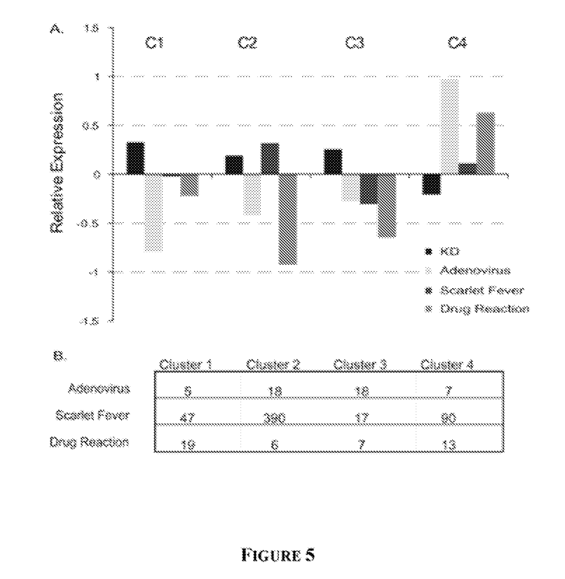

The fourth cluster (C4) consisted of 60 clones (43 genes) that were expressed at lower levels in the KD patients than adenovirus patients. The defining feature of this gene cluster was the presence of canonical interferon-induced genes such as MX1, MX2, ISG15 (G1P2), IFIT2, OAS1 and OAS2. As a group, cluster 4 was highly enriched for genes expressed after stimulation with type I Interferons both in vitro, and in patients with hepatitis C treated with pegylated interferon (all p<10⁻¹⁵). To verify this dramatic difference in interferon-induced gene expression in a separate group of patients, levels of three of these IFN-induced transcripts were measured by RT-PCR in an independent cohort of 10 KD patients, 12 adenovirus infection patients, and 8 healthy controls, matched for age and sex. Levels of these transcripts in the KD patients were again lower than in the adenovirus patients, and similar to levels found in the healthy children (FIG. 5).

### KD and Adenovirus Infection can be Distinguished by Transcript Levels

The presence of multiple expression programs that differed between adenovirus infection patients and KD patients suggested that it might be possible to distinguish them on the basis of a subset of these transcripts. Prediction Analysis of Microarrays (PAM) was used to identify a set of genes that might differentiate between these cohorts. Using a set of 38 genes, and 10-fold cross-validation, the study correctly identified 21 of 23 KD patients, while mis-classifying only 1 of 7 adenovirus infection patients. Sixteen genes were among the interferon-induced gene set, while the other 22 were distributed among the gene sets associated with cell adhesion/motility and innate immune responses. The most robust among the interferon-associated genes were MX1, MX2, IFI2, ISG15, LY6E, OAS1, OAS2, OAS3, IRF2, and IFI27, all of which were expressed at higher levels in adenovirus-infected patients.

An average expression of gene clusters were shown in FIG. 5A. FIG. 5B provided an estimated number of samples (both KD and control) needed to identify an existing difference in expression levels between patients with KD and a specific control group, given the average difference in expression observed in this data set. Cluster numbers correspond to cluster numbers discussed above. The type I error (α) was set at 0.05; power (β) at 0.8.

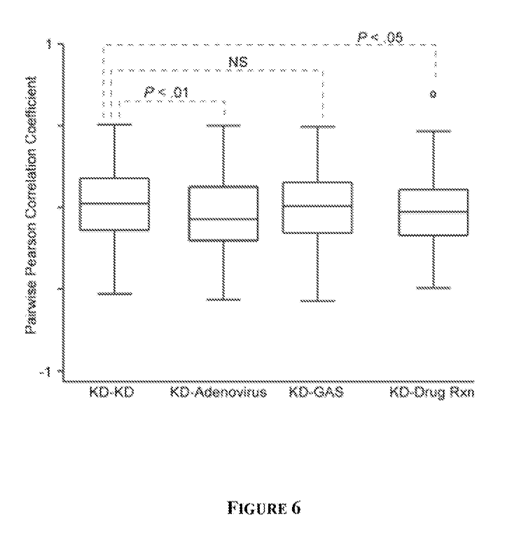

FIG. 6 illustrates a comparison of whole-blood transcription profiles from patients with Kawasaki disease (KD) and control groups. Correlation coefficients were calculated for each KD sample and each control sample in the data set, and they were plotted by comparison group. The central horizontal line in each box represents the median correlation coefficient, the upper and lower borders of each box indicate the 25^{th}-75^{th} percentiles, and the whiskers indicate the 10^{th}-90^{th} percentiles.

### Example 3

### Inflammatory Biomarkers as Indicators in Acute Kawasaki Disease (KD)

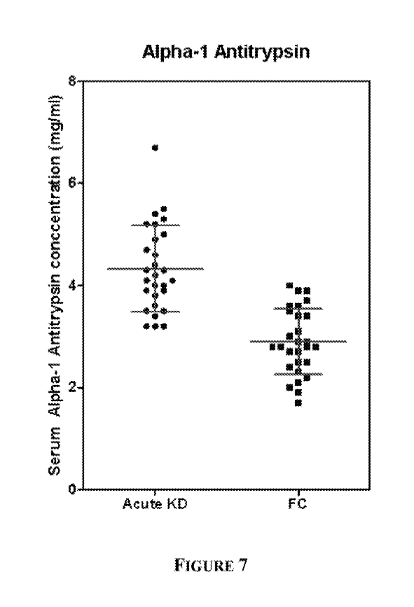

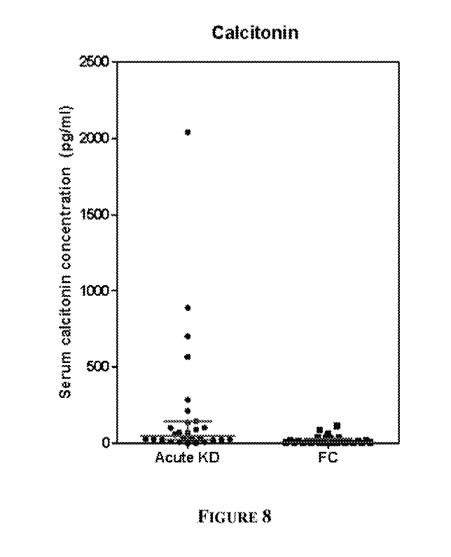

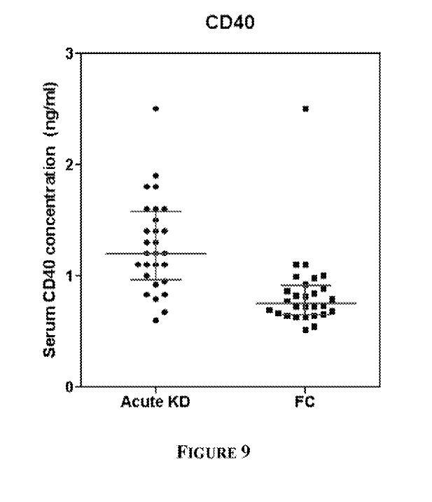

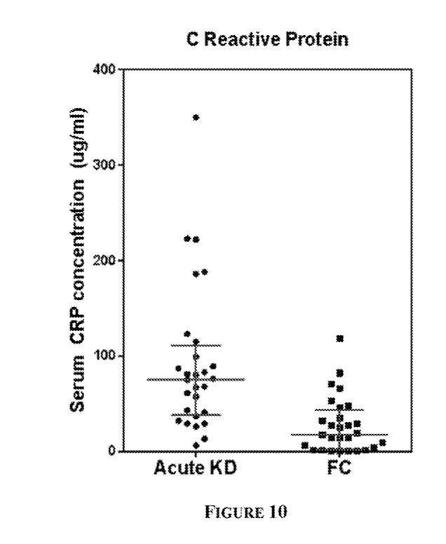

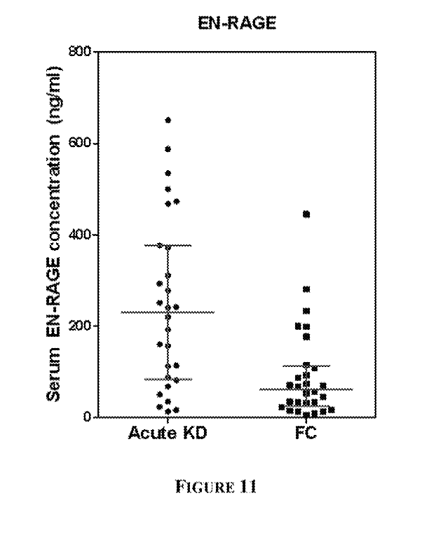

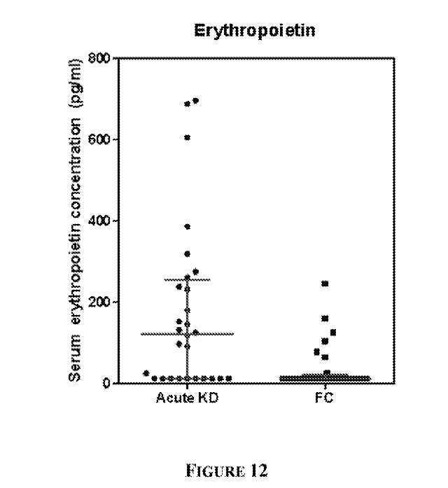

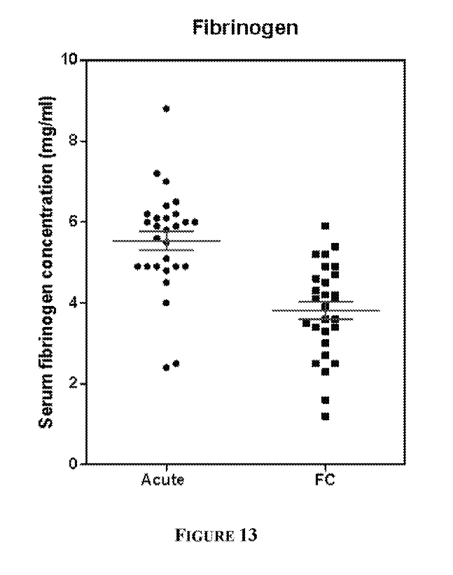

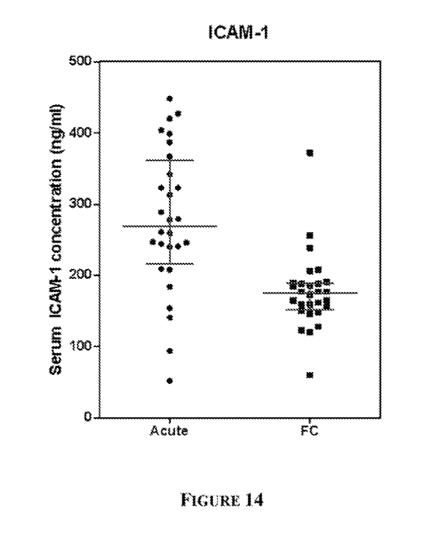

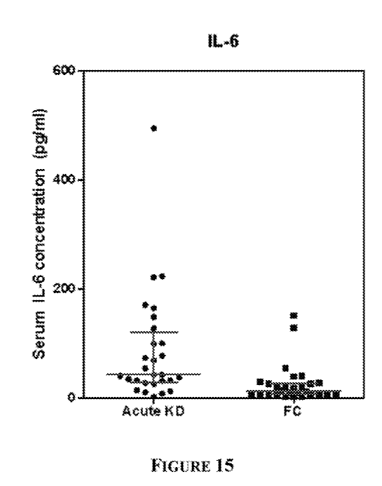

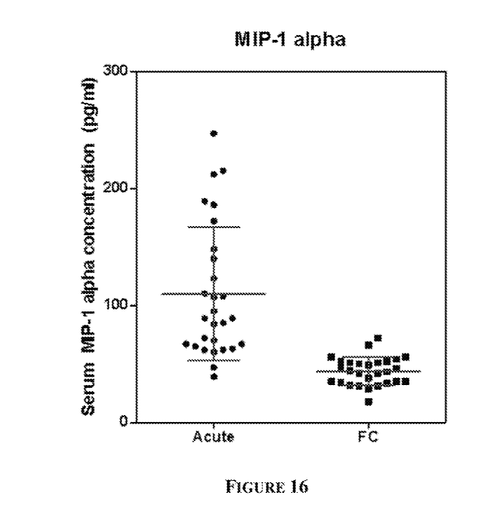

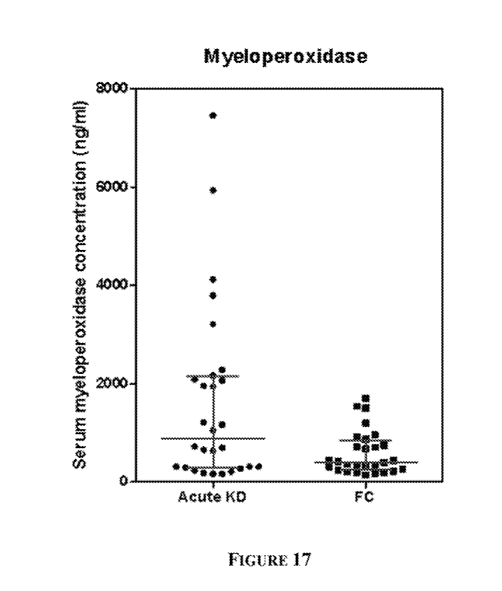

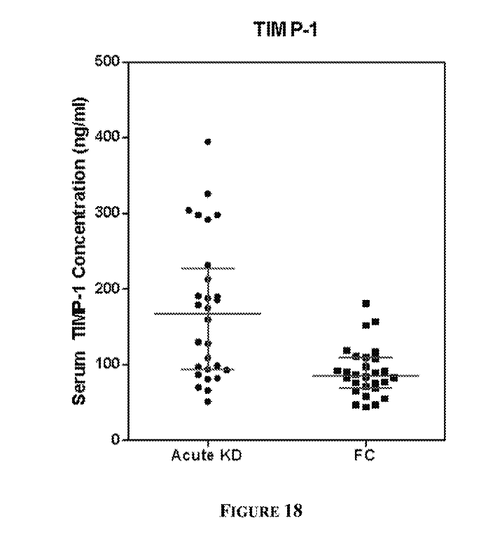

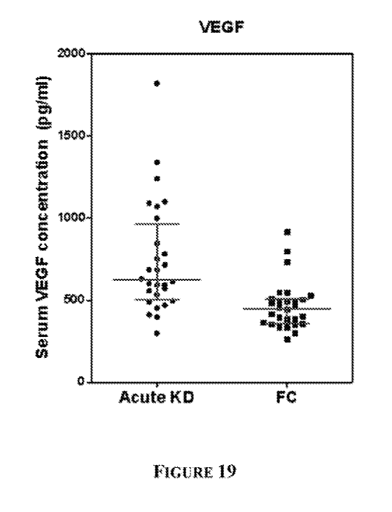

Plasma samples from twenty-eight (28) acute Kawasaki Disease subjects and twenty-eight (28) age- and sex-matched febrile control children were analyzed using the Luminex bead system (RulesBasedMedicine, Inc.) for eighty-nine (89) analytes in pathways related to inflammation. Of the eighty-nine (89) analytes, thirteen (13) analytes showed significantly different levels between acute Kawasaki Disease and febrile control subjects. The median and 95% CI for the two groups are shown for each analyte in these graphs. (FIGS. 7-19).

The thirteen analytes showing significantly different levels in acute Kawasaki Disease are alpha-1 antitrypsin, calcitonin, CD40, C reactive protein, EN-RAGE, erythropoietin, fibrinogen, ICAM-1, IL-6, MIP-1 alpha, myeloperoxidase, TIMP-1, and VEGF.

What is claimed:

1. A Method for diagnosing Kawasaki Disease in a patient comprising:
a) detecting expression levels of at least two Kawasaki Disease diagnostic biomarkers in a biological sample from the patient, wherein the first biomarker is a cardiomyocyte biomarker, and the second biomarker is an inflammatory biomarker, and
b) diagnosing the patient as having Kawasaki Disease when the expression levels of the at least two diagnostic biomarkers in the patient sample are higher than normal expression levels of the same biomarkers derived from a biological sample from a control subject without Kawasaki Disease.

2. The method of claim 1, wherein the cardiomyocyte biomarker is selected from the group consisting of N-terminal pro-B-type natriuretic peptide (NT-proBNP), C-type natriuretic peptide (CNP), atrial natriuretic peptide (ANP), high-sensitivity Troponin, IL-33, or ST.

3. The method of claim 2, wherein the cardiomyocyte biomarker is N-terminal pro-B-type natriuretic peptide (NT-proBNP).

4. The method of claim 1, wherein the cardiomyocyte biomarker is a cardiac biomarker ST2.

5. The method of claim 1, wherein the inflammatory biomarker is selected from the group consisting of a biomarker of endothelial cell, platelet, and leukocyte damage, activation and adhesion, a biomarker of macrophage monocytes, and a biomarker of acute phase reactants.

6. The method of claim 5, wherein the biomarker of endothelial cell, platelet, and leukocyte damage, activation and adhesion is selected from the group consisting of vascular endothelial growth factor (VEGF), CD40, intercellular adhesion molecule-1 (ICAM-1), vascular cell adhesion molecule-1 (VCAM-1), carcinoembryonic antigen cell adhesion molecule-1 (CEACAM-1), EN-RAGE, pro-calcitonin, calcitonin, IL-4, myeloperoxidase (MPO), osteoprotegrin (OPG), neutrophil elastase, MMP-8, SPARC, JAM3, SMOX, ITGB5, ITGA2B, endothelin-1, SCUBE1.

7. The method of claim 5, wherein the biomarker of macrophage monocytes is selected from the group consisting of S100, S100A6, S100A8, S100A9, S100A11, S100A12, S100A13, S100P, S100Z, MIP-1 alpha, TIMP-1.

8. The method of claim 5, wherein the biomarker of acute phase reactants is selected from the group consisting of alpha-1 antitrypsin, C-reactive protein, and fibrinogen.

9. The method of claim 1, further comprising detecting an expression level of a third biomarker in a biological sample from the patient, wherein the third biomarker is an interferon type-I biomarker, and wherein diagnosing the patient as having Kawasaki Disease when the expression level of the interferon type-I biomarker is lower in the patient sample than in a sample from a control subject without Kawasaki Disease.

10. The method of claim 1, wherein the patient test sample is a biological fluid selected from the group consisting of whole blood, plasma, serum, tears, saliva, mucous, cerebrospinal fluid, or urine.

11. The method of claim 1, wherein the detection is performed by one or more antibodies that specifically detect the at least two Kawasaki Disease diagnostic biomarkers.

12. The method of claim 11, wherein the antibodies are immobilized on a solid support.

13. A kit for diagnosing Kawasaki Disease in a patient comprising a) a capture reagent comprising one or more detectors specific for at least two Kawasaki Disease diagnostic biomarkers, wherein the first biomarker is a cardiomyocyte biomarker, and the second biomarker is an inflammatory biomarker, b) a detection reagent, and c) instructions for using the kit to diagnose a patient as having Kawasaki Disease when the expression levels of the at least two diagnostic biomarker in a biological sample from the patient are higher than the expression levels of the same biomarkers in a control subject without Kawasaki Disease.

14. The kit of claim 13, wherein the cardiomyocyte biomarker is selected from the group consisting of N-terminal pro-B-type natriuretic peptide (NT-proBNP), C-type natriuretic peptide (CNP), atrial natriuretic peptide (ANP), high-sensitivity Troponin, IL-33, or ST.

15. The kit of claim 14, wherein the cardiomyocyte biomarker is N-terminal pro-B-type natriuretic peptide (NT-proBNP).

16. The kit of claim 13, wherein the cardiomyocyte biomarker is a cardiac biomarker ST2.

17. The kit of claim 13, wherein the inflammatory biomarker is selected from the group consisting of a biomarker of endothelial cell, platelet, and leukocyte damage, activation and adhesion, a biomarker of macrophage monocytes, and a biomarker of acute phase reactants.

18. The kit of claim 17, wherein the biomarker of endothelial cell, platelet, and leukocyte damage, activation and adhesion is selected from the group consisting of vascular endothelial growth factor (VEGF), CD40, intercellular adhesion molecule-1 (ICAM-1), vascular cell adhesion molecule-1 (VCAM-1), carcinoembryonic antigen cell adhesion molecule-1 (CEACAM-1), EN-RAGE, pro-calcitonin, calcitonin, IL-6, IL-4, myeloperoxidase (MPO), osteoprotegrin (OPG), neutrophil elastase, MMP-8, SPARC, JAM3, SMOX, ITGB5, ITGA2B, endothelin-1, SCUBE1.

19. The kit of claim 17, wherein the biomarker of macrophage monocytes is selected from the group consisting of S100, S100A6, S100A8, S100A9, S100A11, S100A12, S100A13, S100P, S100Z, MIP-1 alpha, TIMP-1.

20. The kit of claim 17, wherein the biomarker of acute phase reactants is selected from the group consisting of alpha-1 antitrypsin, C-reactive protein, and fibrinogen.

21. The kit of claim 13, further comprising a capture reagent comprising a detector specific for an interferon type-I biomarker, and instructions for using the kit to diagnose a patient as having Kawasaki Disease when the expression level of the interferon type-I biomarker is lower in patient biological sample than in a control subject without Kawasaki Disease.

22. The kit of claim 13, wherein the patient test sample is a biological fluid selected from the group consisting of whole blood, plasma, serum, tears, saliva, mucous, cerebrospinal fluid, or urine.

23. The kit of claim 13, wherein the detector comprises one or more antibodies that specifically detect the at least two Kawasaki Disease diagnostic biomarkers.

24. The kit of claim 13, wherein the capture reagent is immobilized on a solid support.

25. A device for diagnosing Kawasaki Disease in a patient comprising a) a capture reagent comprising one or more detectors specific for at least two Kawasaki Disease diagnostic biomarkers, wherein the first biomarker is a cardiomyocyte biomarker, and the second biomarker is an inflammatory biomarker, and b) detecting reagents for detecting an expression level of the at least two diagnostic biomarkers in a biological sample from the patient, wherein the patient is diagnosed as having Kawasaki Disease when the expression levels of the at least two diagnostic biomarkers in the patient biological sample are higher than the expression levels in a control subject without Kawasaki Disease.

26. The device of claim 25, further comprising a capture reagent comprising a detector specific for an interferon type-I biomarker, and detecting reagents for detecting an expression level of the interferon type-I biomarker, wherein the patient is diagnosed as having Kawasaki Disease when the expression level of the interferon type-I biomarker is lower in patient test sample than in a control subject without Kawasaki Disease.
